SEQUENCE LISTING (2 sequences)

SEQ ID NO: 1 - protein, 107 aa, Artificial Sequence
  Description of Artificial Sequence Synthetic polypeptide
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 2 - protein, 119 aa, Artificial Sequence
  Description of Artificial Sequence Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVS

(19) United States
(12) Patent Application Publication
(10) Pub. No.: US 2017/0247443 A1
(43) Pub. Date: Aug. 31, 2017

(54) Safety and Efficacy with a CHO Cell Glycosylated Chimeric Antibody to TNF

(71) Applicants: Jian Cao, San Diego, CA (US); Jeffrey Su, San Diego, CA (US)

(72) Inventors: Jian Cao, San Diego, CA (US); Jeffrey Su, San Diego, CA (US)

(73) Assignee: Sorrento Therapeutics, Inc., San Diego, CA (US)

(21) Appl. No.: 15/403,115

(22) Filed: Jan. 10, 2017

Related U.S. Application Data
(60) Provisional application No. 62/276,954, filed on Jan. 10, 2016.

(51) Int. Cl. C07K 16/24

(52) U.S. Cl. CPC C07K 16/241; C07K 2317/51; C07K 2317/515

(57) ABSTRACT

There is disclosed a chimeric infliximab-like monoclonal antibody having at least 80% NANA glycosylation terminal sialic acid and a glycosylation pattern of Gal-α(2,3/6)-Gal that binds to tumor necrosis factor alpha (TNF). The disclosed infliximab-like monoclonal antibody is a chimeric antibody having the same amino acid sequence (light chain/heavy chain of SEQ ID NO. 1/SEQ ID NO. 2) as infliximab (Remicade®) which has at least 80% NGNA terminal sialic acid and a glycosylation pattern of Gal-α(1,3)-Gal.

### CROSS REFERENCE TO RELATED APPLICATION

This patent application claims priority to U.S. provisional patent application 62/276,954 filed 10 Jan. 2016.

### TECHNICAL FIELD

The present disclosure provides a chimeric infliximab-like monoclonal antibody having at least 80% NANA glycosylation terminal sialic acid and a glycosylation pattern of Gal-α(2,3/6)-Gal. The chimeric infliximab-like monoclonal antibody binds to tumor necrosis factor alpha (TNF) target. The disclosed infliximab-like monoclonal antibody is a chimeric antibody having the same amino acid sequence (light chain/heavy chain of SEQ ID NO. 1/SEQ ID NO. 2) as infliximab (Remicade®). However, the improvement of the present invention is its glycosylation having at least 80% NGNA terminal sialic acid and a glycosylation pattern of Gal-α(1,3)-Gal.

### BACKGROUND

Glycosylation is a post-translational modification. Protein molecular surface sugar chains can have an impact on the structure and function of the protein molecules. Glycosylation and glycan structure of a monoclonal antibody have correlation with its function, by affecting the binding of IgG molecules to FcRs, Clq and FeRn to regulate the antibody-dependent cellular cytotoxicity (ADCC), complement-dependent cytotoxicity (CDC) and half-life of IgG molecules. Glycosylation also affects the safety features of mAb, particularly non-human glycans, and has potential immunogenicity. The glycans located in Fab functional region can affect both the safety and efficacy features of these drugs. Glycosylation is dependent on cell expression system and subclone selection, cell culture factors, such as medium components, and culture conditions. Moreover, glycosylation affects biological activity, efficacy, immunogenicity and pharmacokinetics of therapeutic proteins.

CHO cells and mouse myeloma cells (NS0, SP2/0) expression systems have been used for therapeutic antibody and Fc-fusion proteins. Presently, 48% of currently approved therapeutic monoclonal antibodies are expressed in CHO cells, while 45% are expressed in murine cells (21% NS0 cells, 14% SP2/0 cells, 10% hybridoma cells).

TNF causes pro-inflammatory actions which result in tissue injury, such as inducing procoagulant activity on vascular endothelial cells (Pober et al., J. Immunol. 136:1680 (1986)), increasing the adherence of neutrophils and lymphocytes (Pober et al., J. Immunol. 138:3319 (1987)), and stimulating the release of platelet activating factor from macrophages, neutrophils and vascular endothelial cells (Camussi et al., J. Exp. Med. 166:1390 (1987)).

Drug specific IgE antibodies were found in the serum of most patients with hypersensitivity reactions, and it specifically reacts against α-Gal. Infliximab is expressed and prepared in mammalian cells (mouse myeloma cells SP2/0). This murine cell line contains an additional α1, 3-galactosidase transferase, which primarily mediates the transfer of galactose residue is from UDP-Gal of a conformation to the terminal galactose residues, thereby generating α-Gal. α-Gal is a harmful non-human disaccharide, found in certain glycans on mAb, especially mAb expressed in the murine cell lines. High levels of anti-α-Gal IgE antibodies were found in some patients treated with infliximab. Further, the difference of murine cell IgG glycosylation from human is that, murine cells not only have the biosynthetic machinery to produce α-Gal epitope, but also produce N-hydroxyethyl neuraminidase (NGNA), rather than N-acetyl phenol neuraminidase (NANA). There is an additional oxygen atom in NGNA. Glycoproteins are considered to be closely associated with the immunogenicity in humans if they contain NGNA residues. Some marketed therapeutic glycoproteins have cause serious adverse reactions in the patients because they contains NGNA residues. Therefore, there is a need in the art to improve the safety of infliximab administration by reducing its immunogenicity. The present disclosure was made to improve drug safety.

### SUMMARY

The present disclosure provides a chimeric infliximab-like monoclonal antibody having at least 80% NANA glycosylation terminal sialic acid at an N-glycosylation site and a glycosylation pattern of Gal-α(2,3/6)-Gal. The disclosed infliximab-like monoclonal antibody is a chimeric antibody having the same amino acid sequence (light chain/heavy chain of SEQ ID NO. 1/SEQ ID NO. 2) as infliximab (Remicade®) which has at least 80% NGNA terminal sialic acid and a glycosylation pattern of Gal-α(1,3)-Gal.

In a phase 1 clinical study with healthy volunteers, STI002 showed reduced immunogenic reactions as compared with historical data from infliximab. This observation was confirmed in a multiple dose study in RA (rheumatoid arthritis patients in combination with methotrexate.

### DESCRIPTION OF THE DRAWINGS

FIG. 1 shows a comparison of the disclosed antibody of the disclosed infliximab-like antibody (also called STI002) having similar binding kinetics to infliximab (Remicade®).

FIG. 2 shows a comparison of TNF neutralization and an efficacy or EC50 determination of the disclosed infliximab-like antibody (also called STI002) having similar efficacy to infliximab (Remicade®).

FIG. 3 shows a comparison of TNF neutralization and an efficacy or EC50 determination of the disclosed infliximab-like antibody (also called STI002) having similar efficacy to infliximab (Remicade®).

FIG. 4 shows a comparison of antibody temperature stability of the disclosed infliximab-like antibody (also called STI002) to infliximab (Remicade®).

### DETAILED DESCRIPTION

The invention is based, at least in part, on the therapeutic advantages of producing an anti-TNF antibody in Chinese Hamster Ovary (CHO) cells. STI002 is an anti-TNF antibody that is produced in CHO cells and has the amino acid sequence of infliximab. Structurally, infliximab has a light chain comprising the amino acid sequence set forth in SEQ ID NO: 1, and a heavy chain comprising the amino acid sequence set forth in SEQ ID NO: 2. The amino acid sequences of the infliximab light and heavy chains are described below:

(SEQ ID NO: 1)
Asp Ile Leu Leu Thr Gln Ser Pro Ala Ile Leu Ser
Val Ser Pro Gly Glu Arg Val Ser Phe Ser Cys Arg
Ala Ser Gln Phe Val Gly Ser Ser Ile His Trp Tyr
Gln Gln Arg Thr Asn Gly Ser Pro Arg Leu Leu Ile
Lys Tyr Ala Ser Glu Ser Met Ser Gly Ile Pro Ser
Arg Phe Ser Gly Ser Gly Ser Gly Thr Asp Phe Thr
Leu Ser Ile Asn Thr Val Glu Ser Glu Asp Ile Ala
Asp Tyr Tyr Cys Gln Gln Ser His Ser Trp Pro Phe
Thr Phe Gly Ser Gly Thr Asn Leu Glu Val Lys
SEQ ID NO: 2)
Glu Val Lys Leu Glu Glu Ser Gly Gly Gly Leu Val
Gln Pro Gly Gly Ser Met Lys Leu Ser Cys Val Ala
Ser Gly Phe Ile Phe Ser Asn His Trp Met Asn Trp
Val Arg Gln Ser Pro Glu Lys Gly Leu Glu Trp Val
Ala Glu Ile Arg Ser Lys Ser Ile Asn Ser Ala Thr
His Tyr Ala Glu Ser Val Lys Gly Arg Phe Thr Ile
Ser Arg Asp Asp Ser Lys Ser Ala Val Tyr Leu Gln
Met Thr Asp Leu Arg Thr Glu Asp Thr Gly Val Tyr
Tyr Cys Ser Arg Asn Tyr Tyr Gly Ser Thr Tyr Asp
Tyr Trp Gly Gln Gly Thr Thr Leu Thr Val Ser

Thus, the infliximab-like antibody comprises a light chain comprising the amino acid sequence set forth in SEQ ID NO: 1 and a heavy chain comprising the amino acid sequence set forth in SEQ ID NO: 2. Further, the infliximab-like antibody does not contain either an N-glycolylneuraminic acid (NGNA) glycan or a Gal-α(1,3)-Gal glycan. The infliximab-like antibody does contain glycans associated with CHO cell expression, including, for example, a Gal-α(2, 3/6)-Gal glycan.

The glycosylation mechanism in CHO cells is similar to an IgG glycosylation mechanism in human The present disclosure provides a genetically engineered anti-TNF antibody with different glycan structures than infliximab. By structure analysis, it was determined the infliximab glycan contains primarily α-Gal, and mostly NGNA as the terminal sialic acid. NGNA has very high immunogenicity. At the same time of greatly reduced immunogenicity, the characteristics of the disclosed infliximab-like monoclonal antibody in vivo clearance is in line with the in vivo metabolic of chimeric antibodies, and the pharmacokinetic parameters are consistent with those of infliximab.

Compared with infliximab monoclonal antibody, the disclosed monoclonal antibody has the same amino acid primary structure but does not contain α-Gal. Moreover, the terminal sialic acid is mainly N-acetylneuraminic acid (NANA). These glycosylation changes provide an improvement manifest with better patient tolerance. A clinical study of the disclosed antibody showed better patient tolerance and reduced immunogenicity when compared with published historical data with commercial infliximab. The disclosed monoclonal antibody also showed similar pharmacokinetic in vivo clearance and in vivo metabolism as infliximab according to its commercial product disclosed data.

The present disclosure provides a chimeric monoclonal antibody having at least 80% NANA glycosylation terminal sialic acid at an N-glycosylation site and a glycosylation pattern of Gal-α(2,3/6)-Gal.

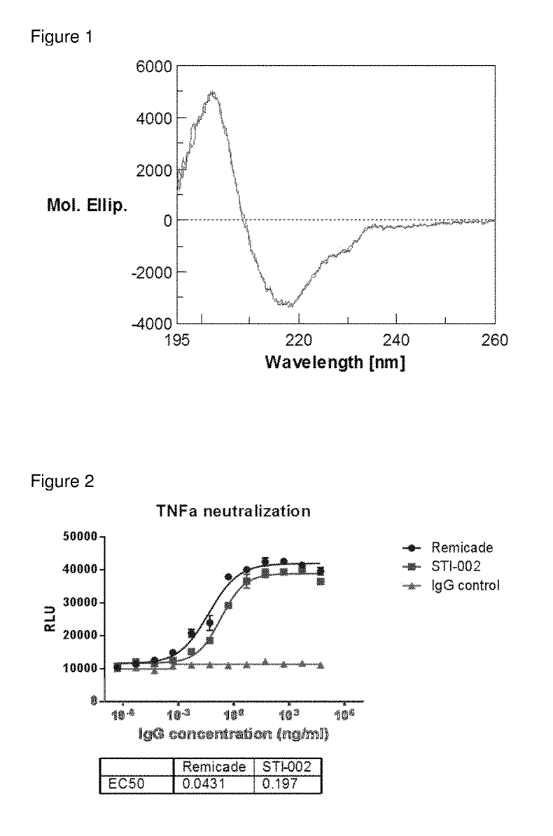

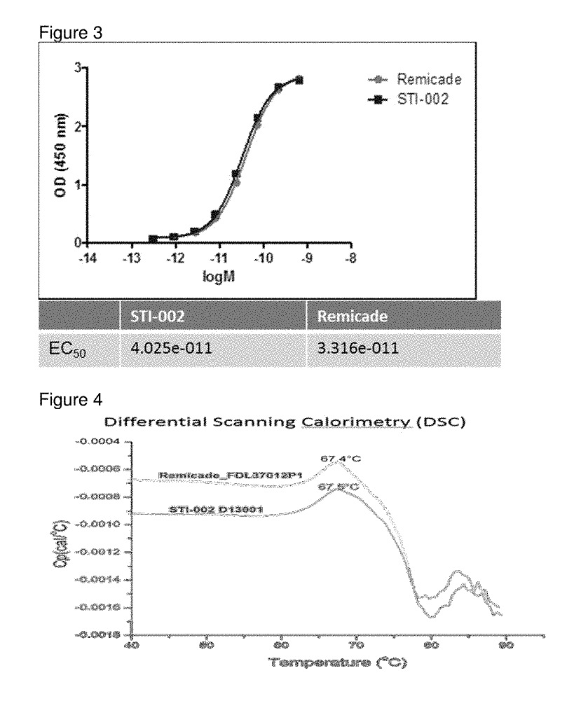

Efficacy was also measured and compared in vitro. FIG. 1 shows a comparison of the disclosed infliximab-like antibody (also called STI002) having similar binding kinetics to infliximab (Remicade®). FIG. 2 shows a comparison of TNF neutralization and an efficacy or EC50 determination of the disclosed infliximab-like antibody (also called STI002) having similar efficacy to infliximab (Remicade®). FIG. 3 shows a comparison of TNF neutralization and an efficacy or EC50 determination of the disclosed infliximab-like antibody (also called STI002) having similar efficacy to infliximab (Remicade®). Both antibodies showed similar efficacy.

1. A pharmaceutical composition comprising an anti-TNF antibody,
wherein the anti-TNF antibody comprises a light chain comprising the amino acid sequence set forth in SEQ ID NO: 1 and a heavy chain comprising the amino acid sequence set forth in SEQ ID NO: 2, and
wherein the anti-TNF antibody comprises a glycosylation pattern having at least 80% NGNA terminal sialic acid and a glycosylation pattern of Gal-α(1,3)-Gal n.

2. The pharmaceutical composition of claim 1, wherein the z-avg of the antibody is 15-20 nm.

3. The pharmaceutical composition of claim 1, wherein the sialic acid glycosylation is at least 80% NANA glycosylation terminal sialic acid at an N-glycosylation site.
